(19) United States
(12) Patent Application Publication
(10) Pub. No.: US 2007/0049833 A1
(43) Pub. Date: Mar. 1, 2007

(54) Arrangements and methods for imaging in vessels

(75) Inventors: Guillermo J. Tearney, Cambridge, MA (US); Milen Shishkov, Watertown, MA (US); Brett Eugene Bouma, Quincy, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation

(21) Appl. No.: 11/505,700

(22) Filed: Aug. 16, 2006

Related U.S. Application Data
(60) Provisional application No. 60/709,088, filed on Aug. 16, 2005.

(51) Int. Cl. A61B 5/05

(52) U.S. Cl. 600/476; 600/407

(57) ABSTRACT

Apparatus, catheter and method for obtaining information regarding a tissue structure (e.g., a blood vessel) can be provided. For example, it is possible to utilize a transceiving arrangement which includes at least one section adapted to be provided in a proximity of at least one portion of the tissue structure and in a bodily fluid, and which is adapted to transmit and receive electromagnetic radiation. The bodily fluid may be blood, pus, necrotic debris, mucus, urine and/or fecal matter.

### CROSS-REFERENCE TO RELATED APPLICATION(S)

This application is based upon and claims the benefit of priority from U.S. Patent Application Ser. No. 60/709,088, filed Aug. 16, 2005, the entire disclosure of which is incorporated herein by reference.

### FIELD OF THE INVENTION

The present invention relates generally to arrangements and methods to be used with anatomical structure, and more specifically for imaging in blood vessels using, e.g., catheters.

### BACKGROUND OF THE INVENTION

Certain research in the field of intravascular imaging has produced an optical diagnostic technology which is capable of analyzing and diagnosing atherosclerotic plaques and other intravascular pathology. Optical coherence tomography (“OCT”) is a recently optical imaging technique capable of obtaining cross-sectional images with a resolution ranging from 2-10 μm. Catheter-based OCT has been used in clinical studies and shown to provide high-resolution images of coronary vascular plaque microstructure. The results indicate that OCT may be used to detect plaques vulnerable to rupture and cause acute myocardial infarction.

One limitation of OCT and other optical imaging methods is their inability to image through blood. Blood has an optical attenuation coefficient similar to tissue and obscures imaging when interposed between the catheter and the vessel wall (as shown in an exemplary cross-sectional image 110 of FIG. 1A). According to recent studies in living animals and patients, OCT imaging of the entire vessel wall was possible primarily by administering a saline purge through the guide catheter to remove blood from the field of view (as shown in an exemplary cross-sectional image 120 of FIG. 1B). While this technique enabled adequate visualization of the vessel wall, unobstructed imaging was possible only for a period of approximately 2-5 seconds. Since the total amount of saline that can be safely administered to a patient is limited, application of this technique would preclude screening of large vessel segments (e.g., greater than 1 mm).

Accordingly, there is a need to overcome the deficiencies as described herein above.

### OBJECTS AND SUMMARY OF EXEMPLARY EMBODIMENTS OF THE INVENTION

To address and/or overcome the above-described problems and/or deficiencies, exemplary embodiments of arrangement (e.g., catheters) can be provided that are designed to allow optical imaging in the vasculature in the presence of blood. These arrangements can overcome the optical attenuation of light by blood by positioning the imaging portion of the catheter near the vessel wall. Imaging may be conducted by pulling back the catheter longitudinally through the vessel lumen. Additional diagnostic information, including pH and temperature, may be obtained by combining the optical imaging probe with other sensor technology. For example, the exemplary embodiments of the present invention can be provided which (i) enable a full optical visualization of the vessel during intravascular imaging in the presence of blood, potentially allowing diagnosis of critical area of interest, (ii) allow OCT screening of large vessel segments, and/or (iii) allow a simultaneous measurement of other clinically relevant parameters including temperature and pH.

Thus, in accordance with one exemplary embodiment of the present invention, an apparatus, catheter and method for obtaining information regarding a tissue structure (e.g., a blood vessel) are provided. For example, it is possible to utilize a transceiving arrangement which includes at least one section adapted to be provided in a proximity of at least one portion of the tissue structure and in a bodily fluid, and which is adapted to transmit and receive electro-magnetic radiation. The bodily fluid may be blood, pus, necrotic debris, mucus, urine and/or fecal matter. The section can be adapted to be provided at approximately at most 6 optical penetration depths of the structure, and may be provided at approximately 250 μm or less from a wall of the structure.

According to another exemplary embodiment of the present invention, the transceiving arrangement can be provided in a housing, and the section of the transceiving arrangement may be extendable from the housing. The section may be a plurality of sections, and each of the sections may be provided at a distinct location in a proximity of the structure. An expandable arrangement (e.g., a spring) can be provided which is adapted to deliver at least one portion of the section in the proximity of the structure. The section can include an optical arrangement which is adapted to deliver at least a portion of the electromagnetic radiation to the structure. The section may be adapted to contact the portion of the tissue. The transceiving arrangement may include a further arrangement which is adapted to translate the at least one section. The section can be translated to distinct locations with respect to the tissue structure so as to obtain data for imaging the tissue structure. The further arrangement is further capable of rotating the section so as to obtain data for imaging the tissue structure. The further arrangement is capable of rotating the section so as to obtain data for imaging the tissue structure. The section may be coupled to a sensing arrangement which is capable of sensing at least one of a temperature, chemical composition or pH level.

These and other objects, features and advantages of the exemplary embodiments of the present invention will become apparent upon reading the following detailed description of embodiments of the invention, when taken in conjunction with the appended claims.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1A is an exemplary OCT image of coronary arteries demonstrates obstruction of view by blood, with only the portion of the vessel in proximity to the catheter sheath is imaged;

FIG. 1B is another exemplary OCT image of coronary arteries demonstrates obstruction of view by blood, with a clear visualization of the entire artery following a saline purge is evident in this OCT image;

FIG. 2 is a schematic illustration of an exemplary embodiment of an optical imaging catheter in accordance with the present invention;

FIG. 3A is a first exemplary embodiment of distal optics arrangement in accordance with the present invention, which may also be angled or cleaved to allow for side-firing illumination and detection;

FIG. 3B is a second exemplary embodiment of distal optics arrangement in accordance with the present invention that has a lens with a single fiber LCI probe;

FIG. 3C is a third exemplary embodiment of distal optics arrangement in accordance with the present invention that has a single fiber LCI probe with a ball lens;

FIG. 4 is a schematic illustration of an exemplary embodiment of a spring-fiber mechanism;

FIG. 5 is a schematic illustration of an exemplary embodiment of an optical imaging catheter for two fibers;

FIG. 6 is a schematic illustration of an exemplary embodiment of an optical imaging catheter having a basket configuration;

FIG. 7 is a schematic illustration of another exemplary embodiment of an optical imaging catheter having the basket configuration with optical fibers and distal optics terminating in a center of the basket;

FIG. 8 is a schematic illustration of still another exemplary embodiment of an optical imaging catheter having the basket configuration in which catheter metallized optical fibers serve as the basket, and the imaging occurs at the center of the basket through cladding/buffer etching; and

FIG. 9 is a schematic illustration of a further exemplary embodiment of the optical imaging catheter that has a thermocouple attached thereto.

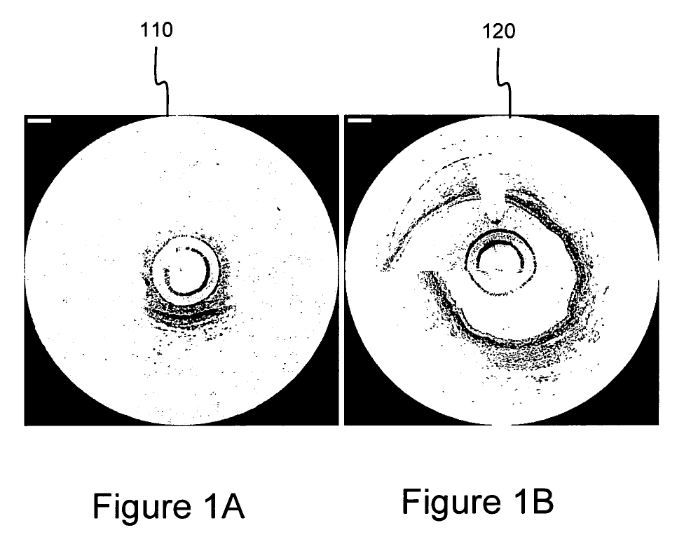

Throughout the figures, the same reference numerals and characters, unless otherwise stated, are used to denote like features, elements, components or portions of the illustrated embodiments. Moreover, while the present invention will now be described in detail with reference to the figures, it is done so in connection with the illustrative exemplary embodiments.

### DETAILED DESCRIPTION OF EXEMPLARY EMBODIMENTS OF INVENTION

Overview

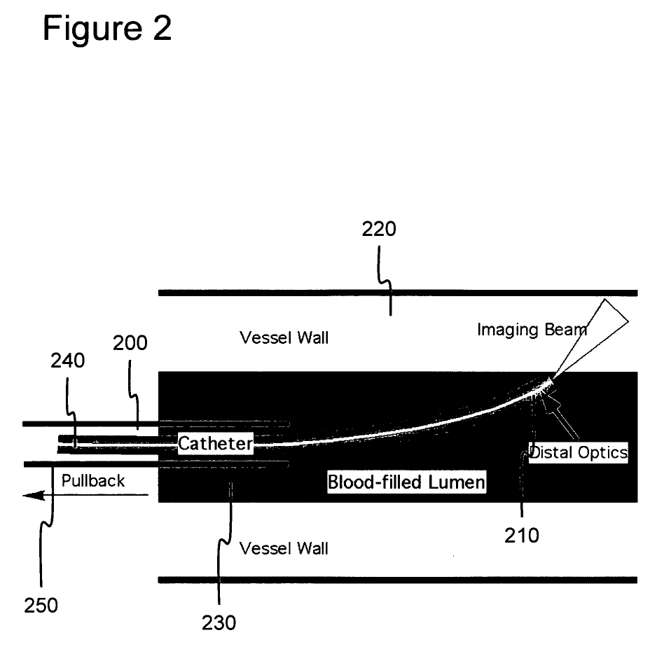

FIG. 2 depicts a schematic illustration of an exemplary embodiment of an optical imaging catheter 200 in accordance with the present invention. For example, the catheter 200 is designed in such a way that the imaging portion 210 of the catheter 200 is placed near to or immediately adjacent to the vessel wall 220, thereby minimizing the optical attenuation of blood 230. In one exemplary embodiment of the present invention, this placement of the distal optics can be effectuated by the mechanical properties or spring mechanism of the catheter. The catheter 200 may be pulled back and/or rotated while the optics are in close contact with the lumen surface and OCT A-lines are obtained for each longitudinal position. In this manner, the imaging in the vessel may be conducted in the presence of blood 230 without a signal attenuation that occurs when imaging through an appreciable blood layer thickness.

Exemplary Deployment

Since, in many instances, the catheter may not be advanced in the expanded position, the catheter 200 may include an inner core 240 and an outer sheath 250. The inner core 240 can be contained within the outer sheath 250 until the catheter 200 is positioned at a location where diagnostic information is to be obtained. Once the catheter 200 is positioned in an appropriate location and/or manner, then the outer sheath 250 may be retracted, thus allowing the spring action of the inner movable component to place the distal optics adjacent to the wall of the catheter 200. The entire catheter 200 (inner and outer components) may be pulled back throughout the blood vessel while optical data is acquired. Alternatively, only the inner component of the catheter 200 may be pulled back within the outer sheath 250.

Exemplary Distal Optics

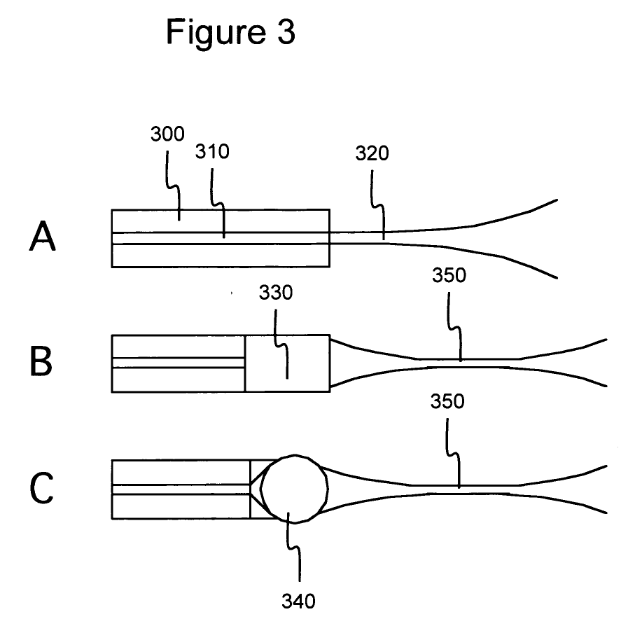

According to one exemplary embodiment of the present invention, the distal optics arrangement of the imaging probe can include a cleaved optical fiber (as shown in FIG. 3A). The optical fiber may be cleaved or polished at an angle to facilitate coupling of the fiber to the tissue surface. As shown in FIG. 3A, the exemplary arrangement can include a cladding 300, a core 310, and a beam waist 320. Other lenses or optical elements may be attached to the fiber to facilitate focusing deeper into the tissue, e.g., including GRIN lens 330 (as shown in another exemplary embodiment of the arrangement of FIG. 3B. Such exemplary embodiment of FIG. 3B includes a cladding 300, a core 310, a lens 330, and a beam waist 350. A ball lens 340 (shown in FIG. 3C illustrating yet another exemplary embodiment of the arrangement), drum lens, microlens, tapered fiber end, prism and the like may also be used. Another exemplary embodiment can includes a sculptured optical fiber tip.

Exemplary Spring action of Inner Core

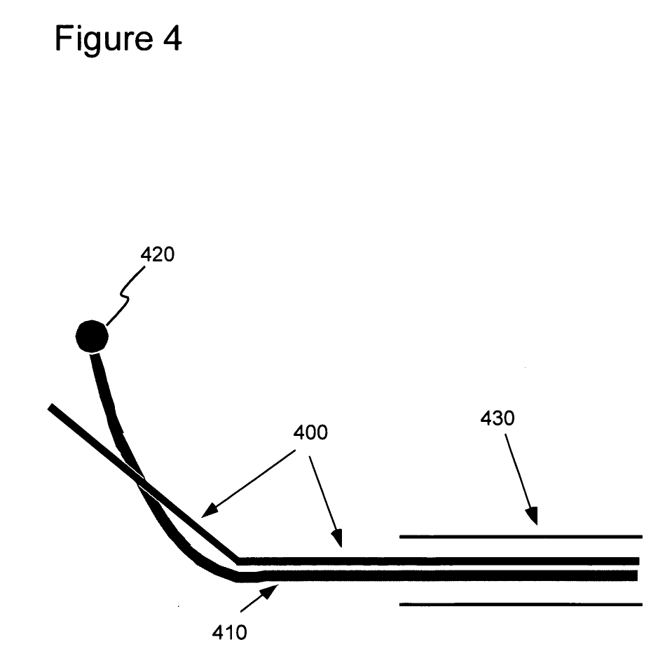

According to further exemplary embodiments of the present invention, the light can be transmitted to and from the vessel wall via the optical fiber. This conduit may be hollow, reflective, and/or contain a step index or a gradient index profile. The optical fiber may be single or multi-mode and may have a single or multiple core. The fiber may act as the inner core if it has mechanical spring action properties that cause it to bend towards the vessel wall. In yet another exemplary embodiment of the present invention, this exemplary spring action may be produced by coating the optical fiber with a metal (e.g. ntinol, gold or other metal) and pre-bending the catheter along the distal portion prior to insertion into the outer sheath. Alternatively or in addition, the optical fiber may be placed in close contact with springs which can reside within the inner sheath. When the outer sheath is retracted, the springs can expand towards the wall, bringing the optical fiber and distal optics near the vessel wall. Such exemplary embodiment and/or operation are shown in FIG. 4, which illustrates an exemplary arrangement that includes a spring 400, an optical fiber 410, distal optics 420 (e.g., a ball lens), and an outer catheter sheath 430 which can perform the above-described exemplary operation.

Exemplary Multiple Fiber Configurations

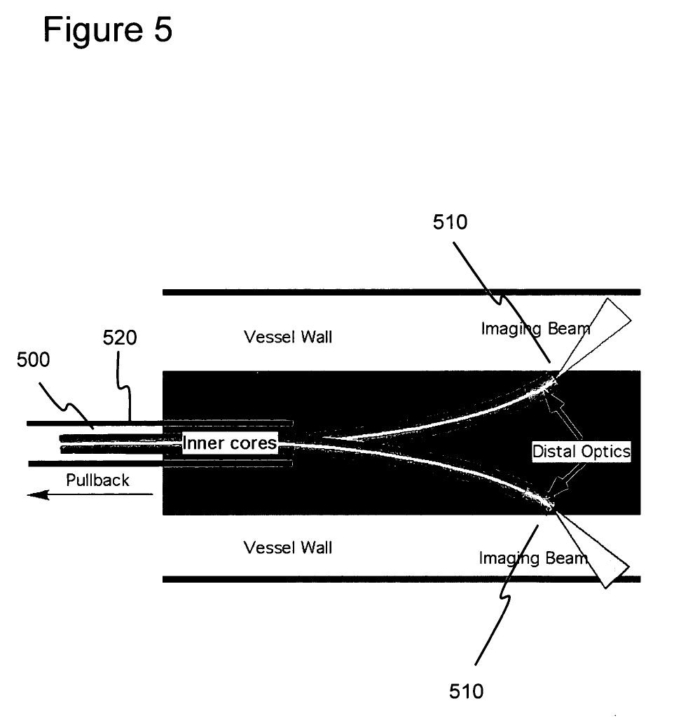

In order to sample more of the lumen, yet another exemplary embodiment of the present invention of the exemplary arrangements can be provided which may include multiple fiber configurations 500 as shown in FIG. 5. For example, certain schematic configuration of an exemplary two-fiber arrangement 500 is illustrated in FIG. 5. The fibers can be configured in a manner such that they approximate the vessel wall 510 at differing locations. As described herein, the multiple fiber configurations may be housed within an external sheath 520 of the exemplary arrangement 500. Other exemplary embodiments of the arrangement can include 3, 4, 5 and 6-fiber configurations.

Exemplary Basket Configurations

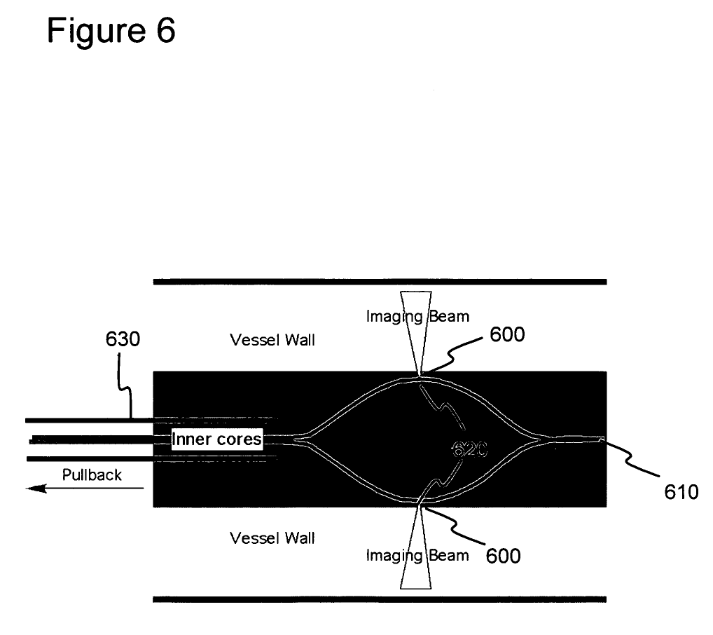

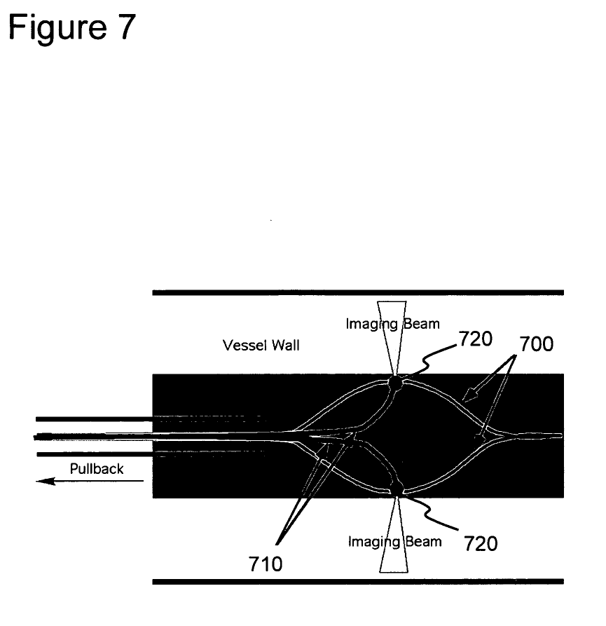

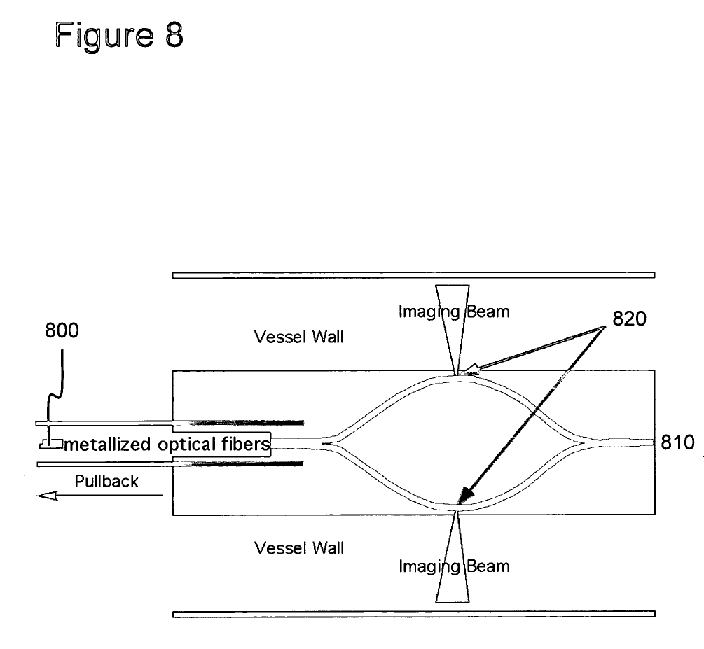

Yet another exemplary embodiment of the arrangement according to the present invention can have a basket configuration as shown in FIG. 6. In this exemplary embodiment of the arrangement, imaging optics 600 are not located at the terminal end of the inner core 610, but within a center of a spring basket 620 that can expand upon its insertion into the vessel or when the outer sheath 630 of the arrangement is retracted. As shown in FIG. 7 which illustrates still another exemplary embodiment of the arrangement according to the present invention, springs 700 may form the entire basket and the optical fiber 710 and distal optics 720 may terminate in the center of the basket. Alternatively or in addition, as shown in FIG. 8 which illustrates yet a further exemplary embodiment of the arrangement according to the present invention, the optical fiber 800 may terminate at the end of the basket 810, and the optical element 820 for delivering and receiving the light may be accomplished by etching the buffer and cladding of the fiber.

Additional Exemplary Diagnostic Probes

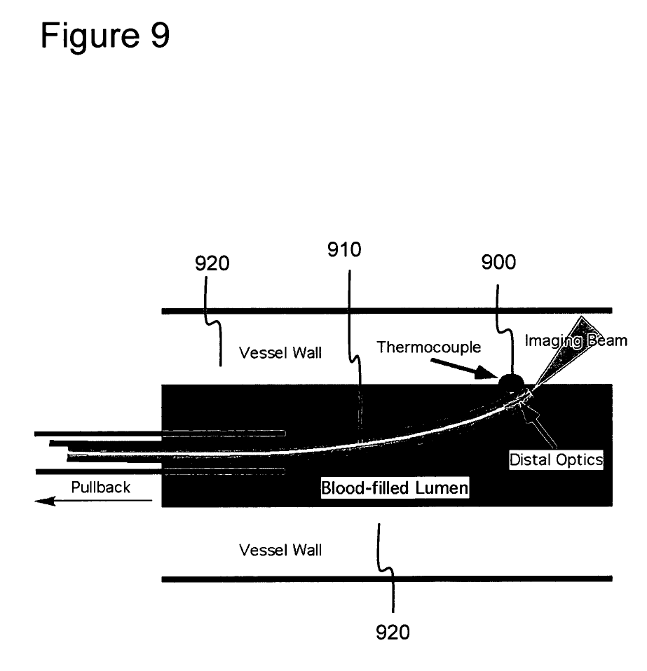

Complementary diagnostic information including temperature, pH, and concentration of biochemicals (e.g. tissue factor) may be obtained by coupling the basket and/or the optical fibers to additional measurement probes. For example, as shown in FIG. 9 which illustrates an additional exemplary embodiment of the arrangement according to the present invention, a thermocouple 900 may be provided and/or attached adjacent to the optical fiber 910 to effectuate or assist with a simultaneous measurement of temperature and an optical signal at the vessel wall 920. Alternatively or in addition, the temperature may be measured from a blackbody infrared radiation using a special (reflective or infrared) waveguide that may be either coupled to the optical fiber. According to another exemplary embodiment of the present invention, the waveguide for the optical measurement may be the same as the waveguide which may be utilized for an infrared temperature measurement. The arterial wall pH may be measured using a fluorescent or absorbing pH indicator at the end of the same or attached optical fiber.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with any OCT system, optical frequency domain imaging (“OFDI”) system, spectral-domain-OCT system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus for obtaining information regarding a tissue structure, comprising:
a transceiving arrangement which includes at least one section adapted to be provided in a proximity of at least one portion of the tissue structure and in a bodily fluid, and which is adapted to transmit and receive electromagnetic radiation.

2. The apparatus of claim 1, wherein the bodily fluid is at least one of blood, pus, necrotic debris, mucus, urine or fecal matter.

3. The apparatus of claim 1, wherein the structure is a blood vessel.

4. The apparatus of claim 1, wherein the at least one section is adapted to be provided at approximately at most 6 optical penetration depths of the structure.

5. The apparatus of claim 1, wherein the at least one section is adapted to be provided at approximately 250 μm from a wall of the structure.

6. The apparatus of claim 1, wherein the transceiving arrangement is provided in a housing, and wherein the at least one section of the transceiving arrangement is extendable from the housing.

7. The apparatus of claim 1, wherein the at least one section is a plurality of sections, and wherein each of the sections is provided at a distinct location in a proximity of the structure.

8. The apparatus of claim 1, further comprising an expandable arrangement which is adapted to deliver at least one portion of the at least one section in the proximity of the structure.

9. The apparatus of claim 8, wherein the expandable arrangement is a spring arrangement.

10. The apparatus of claim 1, wherein the at least one section comprises an optical arrangement which is adapted to deliver at least a portion of the electromagnetic radiation to the structure.

11. The apparatus of claim 1, wherein the at least one section is adapted to contact the at least one portion of the tissue.

12. The apparatus of claim 1, wherein the at least one section is being translated to distinct locations with respect to the tissue structure so as to obtain data for imaging the tissue structure.

13. The apparatus of claim 1, wherein the transceiving arrangement includes a further arrangement which is adapted to translate the at least one section.

14. The apparatus of claim 13, wherein the further arrangement is further capable of rotating the at least one section so as to obtain data for imaging the tissue structure.

15. The apparatus of claim 13, wherein the further arrangement is capable of rotating the at least one section so as to obtain data for imaging the tissue structure.

16. The apparatus of claim 1, wherein the at least one section is adapted to be coupled to a sensing arrangement which is capable of sensing at least one of a temperature, chemical composition or pH level.

17. A method for obtaining information regarding a tissue structure, comprising:
arranging at least one section of a transceiving arrangement in a proximity of at least one portion of the tissue structure and in a bodily fluid; and
transmitting and receive electromagnetic radiation to the tissue structure and from the tissue structure, respectively so as to obtain the information.

18. A catheter, comprising:
a transceiving arrangement which includes at least one section adapted to be provided in a proximity of at least one portion of a tissue structure and in a bodily fluid, and which is adapted to transmit and receive electromagnetic radiation.
